(19) United States
(12) Patent Application Publication
(10) Pub. No.: US 2010/0056858 A1
(43) Pub. Date: Mar. 4, 2010

(54) PACING SYSTEM FOR USE DURING CARDIAC CATHETERIZATION OR SURGERY

(76) Inventors: Eric A. Mokelke, White Bear Lake, MN (US); Allan C. Shuros, St. Paul, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/547,316

(22) Filed: Aug. 25, 2009

Related U.S. Application Data
(60) Provisional application No. 61/190,707, filed on Sep. 2, 2008.

(51) Int. Cl. A61N 1/36; A61B 17/00

(52) U.S. Cl. 600/37; 607/10

(57) ABSTRACT

Cardioprotective pacing is applied to prevent and/or reduce cardiac injury associated with cardiac catheterization or surgery. Pacing pulses are generated from a pacemaker and delivered through one or more pacing electrodes incorporated onto one or more devices used in the cardiac catheterization or surgery. The pacemaker controls the delivery of the pacing pulses by executing a cardioprotective pacing protocol. In one embodiment, the one or more pacing electrodes are incorporated onto an intravascular ultrasound (IVUS) catheter. In another embodiment, the one or more pacing electrodes are incorporated onto one or more cardiac surgical instruments such as a heart stabilizer and a sternal retractor.

### CROSS-REFERENCE TO RELATED APPLICATIONS

This application claims the benefit of U.S. Provisional Application No. 61/190,707, filed on Sep. 2, 2008, under 35 U.S.C. §119(e), which is hereby incorporated by reference in its entirety.

This application is related to co-pending, commonly assigned, U.S. patent application Ser. No. 11/113,828, entitled “METHOD AND APPARATUS FOR PACING DURING REVASCULARIZATION”, filed on Apr. 25, 2005, and U.S. Provisional Patent Application Ser. No. 61/074,066, entitled “EXTERNAL PACEMAKER WITH AUTOMATIC CARDIOPROTECTIVE PACING PROTOCOL”, filed on Jun. 19, 2008, which are hereby incorporated by reference in their entirety.

### TECHNICAL FIELD

This document relates generally to cardiac pacing systems and particularly to a pacing system for delivering cardioprotective pacing using an intravascular ultrasound (IVUS) catheter or cardiac surgical instruments such as heart stabilizer and sternal retractor.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the body organs and pump it to the lungs where the blood gets oxygenated. These pumping functions are resulted from contractions of the myocardium (cardiac muscles). In a normal heart, the sinoatrial node, the heart's natural pacemaker, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract in synchrony to result in efficient pumping functions. A blocked or otherwise abnormal electrical conduction and/or deteriorated myocardial tissue cause dyssynchronous contraction of the heart, resulting in poor hemodynamic performance including a diminished blood supply to the heart and the rest of the body. The condition in which the heart fails to pump enough blood to meet the body's metabolic needs is known as heart failure.

Myocardial infarction (MI) is the necrosis of portions of the myocardial tissue resulted from cardiac ischemia, a condition in which the myocardium is deprived of adequate oxygen supply and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure.

When a blood vessel such as the coronary artery is partially or completely occluded, one ore more catheterization or surgical procedures may be necessary to restore blood supply to the heart. Such procedures may cause ischemic injury in addition to the ischemic injury resulting from MI. Reperfusion as the result of restoration of blood supply is also known to cause cardiac injury, known as reperfusion injury. In general, a cardiac catheterization or surgical procedure may inevitably cause ischemic and/or reperfusion injury of some extent. A substantial percentage of post-operational deaths is reportedly associated with such injury. Therefore, there is a need for minimizing cardiac injury associated with ischemia and reperfusion during cardiac catheterization and surgery.

### SUMMARY

Cardioprotective pacing is applied to prevent and/or reduce cardiac injury associated with cardiac catheterization or surgery. Pacing pulses are generated from a pacemaker and delivered through one or more pacing electrodes incorporated onto one or more devices used in the cardiac catheterization or surgery. The pacemaker controls the delivery of the pacing pulses by executing a cardioprotective pacing protocol.

In one embodiment, the one or more pacing electrodes are incorporated onto an intravascular ultrasound (IVUS) catheter. The IVUS catheter includes a proximal end portion configured to be connected to the pacemaker, a distal end portion configured for intravascular placement, and an elongate shaft connected between the proximal end portion and the distal end portion. The distal portion includes an ultrasonic transducer for ultrasonic imaging and at least one pacing electrode. The pacing pulses are delivered from the pacemaker through that pacing electrode.

In another embodiment, the one or more pacing electrodes are incorporated onto one or more cardiac surgical instruments such as a heart stabilizer and a sternal retractor. The heart stabilizer includes a suction cup and a position arm. The suction cup is configured to hold the heart using vacuum suction and includes at least one pacing electrode. The positioning arm includes a distal end connected to the suction cup, a proximal end including a positioning handle, and an elongate positioning shaft connected between the distal end and the proximal end. The positioning handle is configured to allow a user to manipulate the position of the suction cup by adjusting the shape of the positioning shaft. The pacing pulses are delivered from the pacemaker through the at least one pacing electrode on the suction cup.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a pacing system for use during IVUS catheterization and portions of an environment in which the system is used.

FIG. 2 is an illustration of an embodiment of a pacing system for use during cardiac surgery and portions of an environment in which the system is used.

FIG. 3 is a block diagram illustrating an embodiment of an external pacemaker providing for pacing during cardiac catheterization or surgery.

FIG. 4 is a timing diagram illustrating an embodiment of a cardioprotective pacing protocol.

FIG. 5 is a timing diagram illustrating another embodiment of a cardioprotective pacing protocol.

FIG. 6 is a flow chart illustrating an embodiment of a method for delivering pacing during cardiac catheterization or surgery.

FIG. 7 is a block diagram illustrating an embodiment of an external pacemaker.

FIG. 8 is a block diagram illustrating another embodiment of an external pacemaker.

FIG. 9 is a block diagram illustrating an embodiment of an external pacemaker and electrodes.

FIG. 10 is a block diagram illustrating an embodiment of an external pacemaker and an implantable pacing delivery device.

FIG. 11 is an illustration of an embodiment of exterior configuration of the external pacemaker of FIGS. 7-10.

FIG. 12 is an illustration of another embodiment of exterior configuration of the external pacemaker of FIGS. 7-10.

FIG. 13 is an illustration of an embodiment of exterior configuration of an external device including a pacemaker and a pullback motor.

FIG. 14 is an illustration of another embodiment of exterior configuration of an external device including a pacemaker and a pullback motor.

FIG. 15 is an illustration of an embodiment of an IVUS catheter with pacing electrodes.

FIG. 16 is an illustration of another embodiment of an IVUS catheter with pacing electrodes.

FIG. 17 is an illustration of an embodiment of a catheterization device assembly including an IVUS catheter and a PTVI device each including pacing electrodes.

FIG. 18 is an illustration of an embodiment of a heart stabilizer with pacing electrodes.

FIG. 19 is an illustration of another embodiment of a heart stabilizer with pacing electrodes.

FIG. 20 is an illustration of another embodiment of a heart stabilizer with pacing electrodes.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their legal equivalents.

It should be noted that references to “an”, “one”, or “various” embodiments in this disclosure are not necessarily to the same embodiment, and such references contemplate more than one embodiment.

In this document, “revascularization” includes reopening of a completely or partially occluded blood vessel using percutaneous transluminal vascular intervention (PTVI) procedure, such as a percutaneous transluminal coronary angioplasty (PTCA) procedure performed in response to cardiac ischemia or myocardial infarction (MI). “Cardiac catheterization” includes the PTVI procedure in which a PTVI device provides for access to the heart, including blood vessels on the heart. Examples of a PTVI device include guide wires, guide catheters, angioplasty catheters, and intravascular ultrasound (IVUS) catheters used during mechanical revascularization procedures. “Cardiac surgery” includes surgeries on the heart and/or cardiac vessels, including open-chest surgery (also known as open heart surgery). Examples of cardiac surgery includes coronary artery bypass grafting (CABG), valve replacement, and heart transplant.

This document discusses a pacing system that delivers pacing pulses using one or more pacing electrodes incorporated onto one or more devices used in the cardiac catheterization or surgery. In one embodiment, the one or more pacing electrodes are incorporated onto an IVUS catheter. In another embodiment, the one or more pacing electrodes are incorporated onto one or more cardiac surgical instruments such as a heart stabilizer and a sternal retractor. The pacing system provides for acute pacing cardioprotection therapy without substantially interfering with other procedures of the cardiac catheterization or surgery. The pacing system includes a pacemaker that controls delivery of the acute pacing cardioprotection therapy by executing a cardioprotective pacing protocol specifying a pacing sequence including alternating pacing and non-pacing periods, or alternating pacing modes. The cardioprotective pacing protocol specifies pacing parameters selected to create or augment mechanical stress on the myocardium or particular regions of the myocardium. In one embodiment, the pacemaker is an external pacing device such as a pacing system analyzer (PSA).

In various embodiments, incorporation of pacing electrodes into IVUS catheters, other PTVI devices, and/or cardiac surgical instruments allow timely delivery of cardioprotective pacing therapy during cardiac catheterization or surgical procedures that are known to associate with ischemic and reperfusion injuries. In various embodiments, cardioprotective pacing therapies delivered using one or more devices discussed in this documents include pacing pre-conditioning therapy that is delivered to minimize injury associated with an anticipated ischemic or reperfusion event and pacing post-conditioning therapy that is delivered to minimize injury associated with an ischemic or reperfusion event that has occurred.

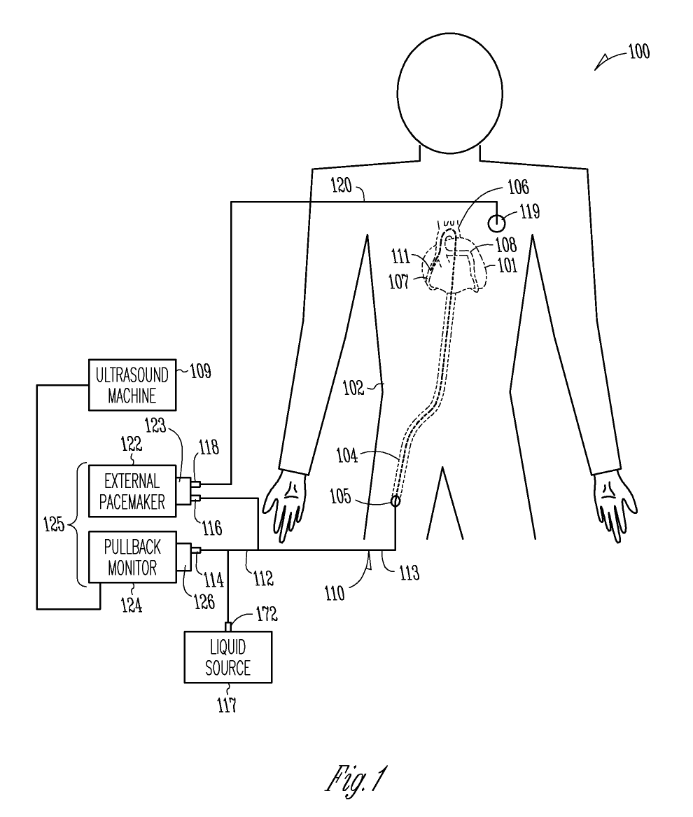

FIG. 1 is an illustration of an embodiment of a pacing system 100 for use during IVUS catheterization and portions of an environment in which system 100 is used. System 100 includes an IVUS catheter 110 connected to an external pacemaker 122, a pullback motor 124, an ultrasound machine 109, and a liquid source 117. One or more pacing electrodes are incorporated onto IVUS catheter 110 for delivering pacing during IVUS catheterization. When needed, system 100 also includes a reference electrode 119, which is a body-surface electrode, such as a skin patch electrode, connected to a lead 120. Lead 120 is connected to a connector 118 allowing its connection to external pacemaker 122.

IVUS catheter 110 is used for intravascular imaging during a revascularization procedure and includes a distal end portion 111 for intravascular placement, a proximal end portion 112, and an elongate shaft 113 coupled between distal end portion 111 and proximal end portion 112. Proximal end portion 112 includes various connectors and other structures allowing manipulation of IVUS catheter 110 including the percutaneous transluminal insertion of the device and operation of an ultrasound transducer at distal end 111. The illustrated connectors include a transducer connector 114, a pacing connector 116, and an injection port 172. Pullback motor 124 drives the ultrasound transducer and includes a motor connector 126 that mates transducer connector 114. External pacemaker 122 delivers pacing pulses through the one or more pacing electrodes incorporated onto IVUS catheter 110 and includes a pacemaker connector 123 that includes one or more connectors mating pacing connector 116 and connector 118. Liquid source 117 allows intravascular administration of a liquid such as a therapeutic or imaging agent via IVUS catheter 110.

In the illustrated embodiment, IVUS catheter 110 is used in a PTCA procedure. During the PTCA procedure, an opening 105 is made on a femoral artery 104 in a patient's body 102. IVUS catheter 110 is inserted into femoral artery 104 and advanced to an aorta 106 and then to a right coronary artery 107, which is narrowed or blocked. The ultrasonic transducer at distal end 111 is then used to visualize the interior of right coronary artery 107. In another embodiment, IVUS catheter 110 is used to visualize the interior of a blocked left coronary artery 108.

Distal end portion 111 of IVUS catheter 110 includes the one or more pacing electrodes to allow pacing pulses to be delivered to a heart 101 during the PTCA procedure. In one embodiment, pacing pulses are delivered through two pacing electrodes on distal end portion 111 of IVUS catheter 110. In another embodiment, pacing pulses are delivered through a pacing electrode on distal end portion 111 of IVUS catheter 110 and surface electrode 119 functioning as the return electrode for pacing. In another embodiment, pacing pulses are delivered through a pacing electrode on distal end portion 111 of IVUS catheter 110 and a pacing electrode on another PTVI device such as a guide wire used for inserting IVUS catheter 110.

External pacemaker 122 delivers pacing pulses by executing a cardioprotective pacing protocol. In one embodiment, the cardioprotective pacing protocol specifies a cardioprotective pacing sequence for preventing arrhythmia and cardiac injuries associated with the revascularization procedure. In one embodiment, pacemaker 122 is an external pacemaker such as a PSA. In another embodiment, pacemaker 122 includes an implantable pacemaker adapted for external use.

Ultrasound machine 109 produces ultrasound images using signals acquired by IVUS catheter 110. An example of ultrasound machine 109 is the iLab® Ultrasound Imaging System provided by Boston Scientific Corporation (Natick, Mass. 01760). In one embodiment, IVUS catheter 110 is a pacing catheter formed by incorporating the one or more pacing electrodes onto an IVUS catheter such as the Atlantis® SR Pro Coronary Imaging Catheter provided by Boston Scientific Corporation.

It is to be understood that FIG. 1 is for illustrative, but not restrictive, purposes. For example, the physical structure of proximal end portion 112 depends on functional and ease-of-use considerations. In one embodiment, external pacemaker 122 and pullback motor 124 are integrated into an external device 125. In another embodiment, external pacemaker 122 and pullback motor 124 are separate devices. In one embodiment, transducer connector 114 and pacing connector 116 branch out from shaft 113, as illustrated in FIG. 1. In another embodiment, transducer connector 114 and pacing connector 116 are integrated into one catheter connector, and motor connector 126 and pacemaker connector 123 are also integrated into one external device connector. In one embodiment, IVUS catheter 110 includes one or more electrodes suitable for delivering cardioversion/defibrillation pulses, in addition to delivering the pacing pulses.

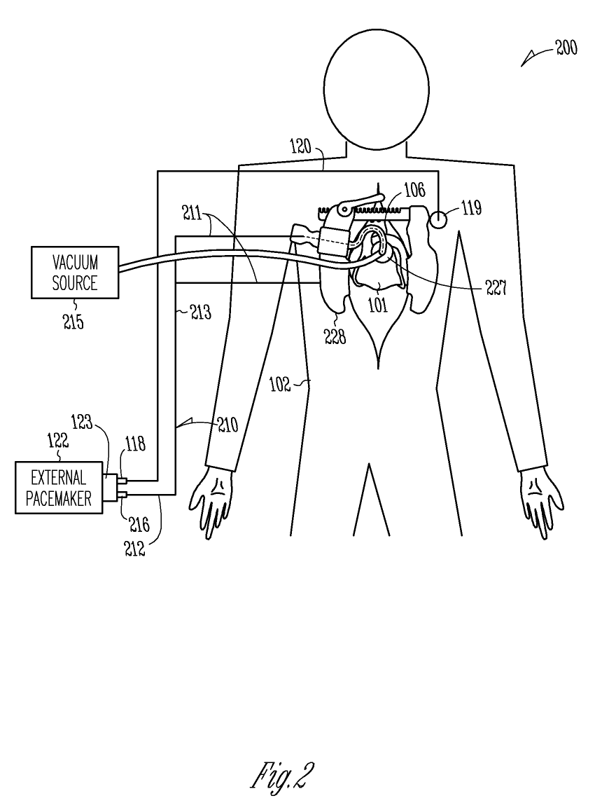

FIG. 2 is an illustration of an embodiment of a pacing system 200 for use during cardiac surgery and portions of an environment in which system 200 is used. System 200 includes one or more cardiac surgical instruments including one or more pacing electrodes, and a pacing lead 210 connected between the one or more pacing electrodes and external pacemaker 122. When needed, system 200 also includes reference electrode 119 connected to external pacemaker 122 via lead 120.

The one or more cardiac surgical instruments are used during a cardiac surgery such as CABG, valve replacement, and heart transplant. In one embodiment, the one or more pacing electrodes are incorporated into one or more instruments of the ACROBAT® V Vacuum Off-Pump System, provided by Boston Scientific Corporation, for use in “off-pump” cardiac surgery. An “off pump” surgery is performed while the heart is beating, without using a heart-lung machine (“pump”) to provide cardiopulmonary bypass. In the illustrated embodiment, the one or more cardiac surgical instruments include a heart stabilizer 227 and a sternal retractor 228. Heart stabilizer 227 provides for stabilizing and manipulating the position of heart 101 while it is beating during the cardiac surgery. It holds heart 101 using vacuum suction provided by a vacuum source 215. An example of heart stabilizer 227 includes one or more pacing electrodes incorporated onto a heart stabilizer such as the EPOSE® 3 Access Device provided by Boston Scientific Corporation. The one or more pacing electrodes are located on heart stabilizer 227 to contact heart 101 during the cardiac surgery. Sternal retractor 228 provides for retracting the rib cage, thereby keeping the chest open and heart 101 exposed during the cardiac surgery. In various embodiments, sternal retractor 228 includes at least a portion functioning as a pacing electrode. In one embodiment, pacing pulses are delivered through two pacing electrodes on heart stabilizer 227. In another embodiment, pacing pulses are delivered through a pacing electrode on heart stabilizer 227 and surface electrode 119 functioning as the return electrode for pacing. In another embodiment, pacing pulses are delivered through a pacing electrode on heart stabilizer 227 and sternal retractor 228.

Pacing lead 210 includes a distal end 211, a proximal end 212, and an elongate lead body 213 coupled between distal end 211 and proximal end 212. Distal end 211 is connected to the one or more pacing electrodes. Proximal end 212 includes a pacing connector 216 that mates pacemaker connector 123.

It is to be understood that FIG. 2 is for illustrative, but not restrictive, purposes. For example, in various embodiments, the one or more pacing electrodes are incorporated into any one or more surgical instruments that provide for direct and stable contact with heart 101 when pacing is to be delivered during the cardiac surgery. In one embodiment, the one or more surgical instruments also include one or more electrodes suitable for delivering cardioversion/defibrillation pulses.

### External Pacemaker

FIG. 3 is a block diagram illustrating an embodiment of an external pacemaker 322 that provides for pacing during cardiac catheterization or surgery. External pacemaker 322 is an embodiment of pacemaker 122 and includes a pacing output circuit 330, a user interface 334, and a control circuit 332. Pacing output circuit 330 delivers pacing pulses to IVUS catheter 110. User interface 334 allows a user to control the delivery of the pacing pulses by controlling pacing parameters and/or timing of the delivery. Control circuit 332 controls the delivery of the pacing pulses. In one embodiment, external pacemaker 322 is a PSA including a chassis that houses pacing output circuit 330 and control circuit 332. User interface 334 is incorporated onto the chassis.

In the illustrated embodiment, control circuit 332 includes a pacing protocol module 335, which enables control circuit 332 to control the delivery of the pacing pulses by automatically executing a pacing protocol. To provide an acute pacing cardioprotection therapy, the pacing protocol specifies a cardioprotective pacing sequence that includes alternating pacing and non-pacing periods or alternating pacing modes for delivering pacing during cardiac catheterization or surgery.

In one embodiment, pacing protocol module 335 is configured to be detachably connected to external pacemaker 322. In a specific embodiment, pacing protocol module 335 includes a memory device that stores the cardioprotective pacing protocol, and control circuit 332 is capable of automatically executing the cardioprotective pacing protocol when pacing protocol module 335 is connected to external pacemaker 322. In another specific embodiment, in addition to the memory device that stores the cardioprotective pacing protocol, pacing protocol module 335 includes a user interface that allows the user to adjust parameters of the cardioprotective pacing protocol and/or control circuitry that supplement the functions of control circuit 332 for automatically executing the cardioprotective pacing protocol. In various embodiments, other pacing protocol modules are provided for automatically executing pacing protocols using external pacemaker 322. In various embodiments, the user is provided with external pacemaker 322 and pacing protocol modules for executing pacing protocols such as the cardioprotective pacing protocol, cardiac resynchronization therapy (CRT) pacing protocol, and cardiac remodeling control therapy (RCT) pacing protocol. Compared to a PSA that requires the user to manually adjust pacing parameters during a test or therapy session, the automatic execution of the pacing protocol increases the accuracy of pacing control and reduces or eliminates the need for the user to control the delivery of the pacing pulses, so that the user can be more attentive to the response of the patient and/or the cardiac catheterization or surgery procedure.

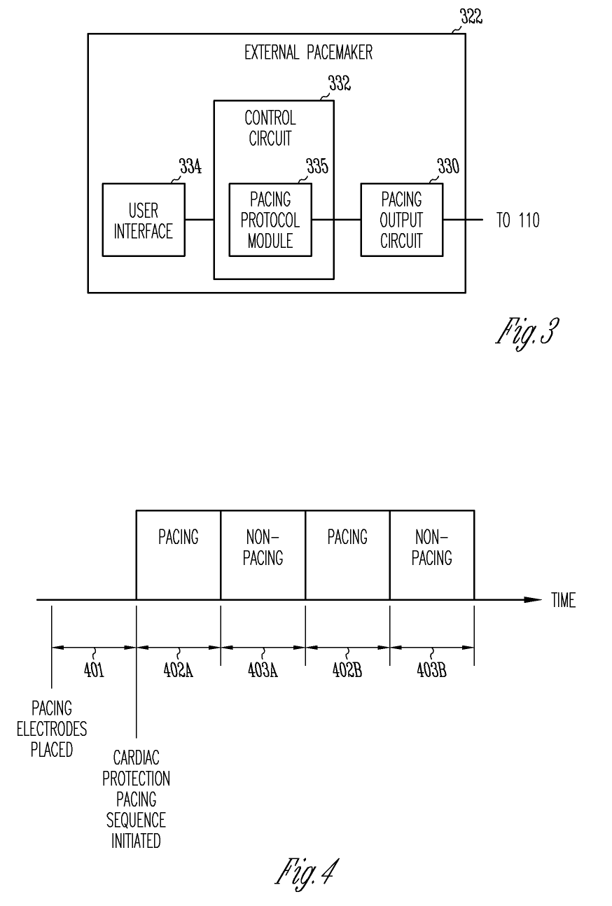

FIG. 4 is a timing diagram illustrating an embodiment of the cardioprotective pacing protocol that specifies a cardioprotective pacing sequence. The cardioprotective pacing sequence is initiated after a time interval 401 that starts when pacing electrodes are placed in desirable locations in and/or on body 102. Time interval 401 expires before, during, and/or after an ischemic or reperfusion event. In one embodiment, the cardioprotective pacing sequence is applied repeatedly, before, during, and/or after the ischemic or reperfusion event.

As illustrated in FIG. 4, the cardioprotective pacing sequence includes alternating pacing and non-pacing periods. Each pacing period is a pacing duration during which the pacing pulses are delivered in a predetermined pacing mode. The non-pacing period is a non-pacing duration during which no pacing pulses is delivered. In one embodiment, during each pacing period, rapid, asynchronous pacing is applied. In other words, pacing pulses are delivered at a rate substantially higher than the patient's intrinsic heart rate without being synchronized to the patient's intrinsic cardiac contractions. For illustrative purpose only, FIG. 4 shows a cardioprotective pacing sequence that includes two cycles of alternating pacing and non-pacing periods: pacing period 402A, non-pacing periods 403A, pacing period 402B, and non-pacing periods 403B. In one embodiment, the number of the cycles of alternating pacing and non-pacing periods is programmable, and each of the pacing and non-pacing periods is programmable. In one embodiment, the cardioprotective pacing sequence is initiated before the ischemic or reperfusion event and includes approximately 1 to 4 cycles of alternating pacing and non-pacing periods. The pacing period is in a range of approximately 30 seconds to 20 minutes. The non-pacing period is in a range of approximately 30 seconds to 20 minutes. In a specific example, the cardioprotective pacing sequence initiated before the ischemic or reperfusion event includes 3 cycles of alternating pacing and non-pacing periods each being approximately 5-minute long. In one embodiment, the cardioprotective pacing sequence is initiated during the ischemic or reperfusion event and includes approximately 1 to 4 cycles of alternating pacing and non-pacing periods. The pacing period is in a range of approximately 30 seconds to 20 minutes. The non-pacing period is in a range of approximately 30 seconds to 20 minutes. In a specific example, the cardioprotective pacing sequence delivered during the ischemic or reperfusion event includes 3 cycles of alternating pacing and non-pacing periods each being approximately 5-minute long. In one embodiment, the cardioprotective pacing sequence is initiated after the ischemic or reperfusion event and includes approximately 1 to 4 cycles of alternating pacing and non-pacing periods. The pacing period is in a range of approximately 10 seconds to one minute. The non-pacing period is in a range of approximately 10 seconds to one minute. In one specific example, the cardioprotective pacing sequence delivered after the ischemic or reperfusion event includes 2 to 4 cycles of alternating pacing and non-pacing periods each being approximately 30-second long.

In various other embodiments, the cardioprotective pacing sequence includes pacing at one or more atrial tracking or other pacing modes. Examples of pacing modes used in such a cardioprotective pacing sequence include VDD, VVI, and DDD modes. In various embodiments, the VVI and DDD modes are delivered with a lower rate limit higher than the patient's intrinsic heart rate. In one embodiment, pacing therapy is delivered with pacing mode and/or other pacing parameters selected to create or augment mechanical stress on the myocardium or particular regions of the myocardium to a level effecting cardioprotection against myocardial injury using the pacing pulses. In another embodiment, pacing therapy is delivered to prevent restenosis. In another embodiment, pacing therapy is delivered to treat an arrhythmia during the cardiac catheterization or surgery procedure, for example, when the patient experiences bradycardia during the procedure.

In one embodiment, the pacing pulses are delivered according to the cardioprotective pacing protocol through IVUS catheter 110 during the cardiac catheterization or surgery procedure. After the cardiac catheterization or surgery procedure, if an implantable pacemaker is implanted into the patient, pacing therapy is delivered to heart 101 through one or more implantable leads from the implantable pacemaker. The pacing therapy includes delivering pacing pulses according to a pacing sequence that is substantially identical or similar to the cardioprotective pacing sequence applied during the cardiac catheterization or surgery procedure. The pacing sequence is delivered according to a predetermined schedule, such as on a predetermined periodic basis. This prevents or reduces possible cardiac injury after the cardiac catheterization or surgery, including cardiac injury and occurrences of arrhythmia caused by ischemic events including myocardial infarction that may be experienced by the patient after the implantation of the implantable pacemaker.

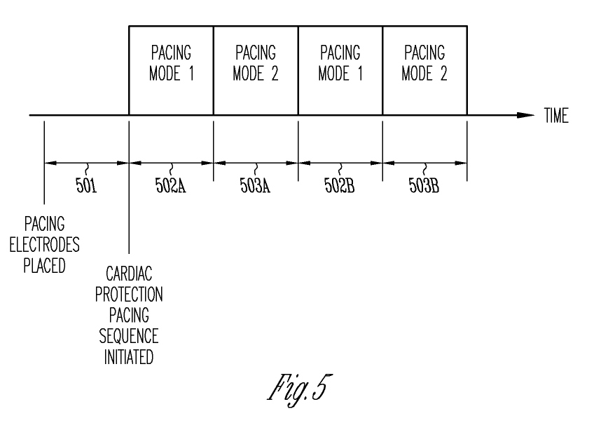

FIG. 5 is a timing diagram illustrating another embodiment of the cardioprotective pacing protocol that specifies a cardioprotective pacing sequence. The cardioprotective pacing sequence is similar to the cardioprotective pacing sequence discussed above with reference to FIG. 4, except that instead of including alternating pacing and non-pacing periods, it includes alternating first and second pacing modes. In various embodiments, the first pacing mode and the second pacing mode substantially differ by at least one pacing parameter value.

The cardioprotective pacing sequence is initiated after a time interval 501 that starts when pacing electrodes are placed in desirable locations in and/or on body 102. Time interval 401 expires before, during, and/or after an ischemic or reperfusion event. In one embodiment, the cardioprotective pacing sequence is applied repeatedly, before, during, and/or after the ischemic or reperfusion event.

As illustrated in FIG. 5, the cardioprotective pacing sequence includes alternating first pacing periods 502A-B and second pacing periods 503A-B. Each pacing period is a pacing duration during which the pacing pulses are delivered in a predetermined pacing mode. First pacing periods 502A-B are each a pacing duration during which pacing pulses are delivered in pacing mode 1. Second pacing periods 503A-B are each a pacing duration during which pacing pulses are delivered according to pacing mode 2.

For illustrative purpose only, FIG. 5 shows a cardioprotective pacing sequence that includes two cycles of alternating first and second pacing periods: first pacing period 502A, second pacing periods 503A, first pacing period 502B, and second pacing periods 503B. In one embodiment, the number of the cycles of the alternating first and second pacing periods is programmable, and each of the first and second pacing periods is programmable. In one embodiment, the cardioprotective pacing sequence is initiated before the ischemic or reperfusion event and includes approximately 1 to 4 cycles of alternating first and second pacing periods. The first pacing period is in a range of approximately 30 seconds to 20 minutes. The second pacing period is in a range of approximately 30 seconds to 20 minutes. In a specific example, the cardioprotective pacing sequence initiated before the ischemic or reperfusion event includes 3 cycles of alternating first and second pacing periods each being approximately 5-minute long. In one embodiment, the cardioprotective pacing sequence is initiated during the ischemic or reperfusion event and includes approximately 1 to 4 cycles of alternating first and second pacing periods. The first pacing period is in a range of approximately 30 seconds to 20 minutes. The second pacing period is in a range of approximately 30 seconds to 20 minutes. In a specific example, the cardioprotective pacing sequence delivered during the ischemic or reperfusion event includes 3 cycles of alternating first and second pacing periods each being approximately 5-minute long. In one embodiment, the cardioprotective pacing sequence is initiated after the ischemic or reperfusion event and includes approximately 1 to 4 cycles of alternating first and second pacing periods. The first pacing period is in a range of approximately 10 seconds to one minute. The second pacing period is in a range of approximately 10 seconds to one minute. In one specific example, the cardioprotective pacing sequence delivered after the ischemic or reperfusion event includes 2 to 4 cycles of alternating pacing and non-pacing periods each being approximately 30-second long.

In various other embodiments, the pacing modes 1 and 2 include atrial tracking and/or other pacing modes. Examples of pacing modes used in such a cardioprotective pacing sequence include VDD, VVI, and DDD modes. In one embodiment, pacing modes 1 and 2 are atrial tracking pacing modes, with a relatively short atrioventricular AV delay used in pacing mode 1 and a relatively long atrioventricular AV delay used in pacing mode 2. In another embodiment, pacing modes 1 and 2 are bradycardia pacing modes, with a relatively high pacing rate used in pacing mode 1 and a relatively low pacing rate used in pacing mode 2. Other pacing modes, including various pacing parameters, are used in various embodiments, depending on patients' needs and conditions.

In various embodiments, a cardioprotective pacing sequence includes either the cardioprotective pacing sequence illustrated in FIG. 4 or the cardioprotective pacing sequence illustrated in FIG. 5.

### Example: External Pacemaker With Automatic Cardioprotective Pacing Protocol

FIG. 6 is a flow chart illustrating of an embodiment of a method 600 for delivering pacing during cardiac catheterization or surgery. Method 600 uses a pacing system executing an automatic pacing protocol specifying times and values for dynamic pacing parameter changes, eliminating the need for manual adjustment of pacing parameters. In various embodiments, the pacing system is connected to one or more of the IVUS catheter and cardiac surgical instruments discussed in this document to deliver pacing pulses through one or more pacing electrodes incorporated onto the one or more of these devices.

Instructions for executing a pacing protocol are stored in a pacing protocol module at 610. The pacing protocol specifies, among other things, a pacing algorithm and its parameters, including timing for changing the parameters. In one embodiment, the pacing protocol is a cardioprotective pacing protocol for delivering pacing during a cardiac catheterization or surgery procedure, such as one of the cardioprotective pacing protocols discussed above with reference to FIG. 4 and FIG. 5. In one embodiment, the cardioprotective pacing protocol is executed to deliver pacing pulses during a revascularization procedure such as a PTCA procedure. Such an acute pacing cardioprotection therapy is applied peri-PTCA procedure to limit the myocardial injury caused by MI and reperfusion, thereby limiting the size of infarcted myocardial tissue in the heart of the patient in whom the revascularization procedure is performed. In another embodiment, the cardioprotective pacing protocol is executed to deliver pacing pulses during a cardiac surgery such as CABG, valve replacement, and heart transplant. Such an acute pacing cardioprotection therapy is applied peri-surgical procedure to limit the myocardial injury caused by ischemia and reperfusion that are inevitably associated with the surgery.

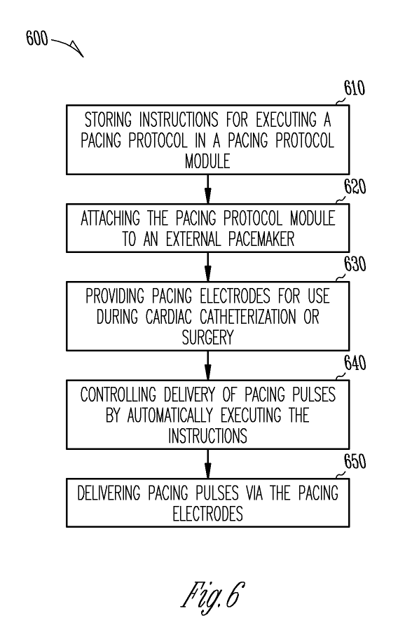

The pacing protocol module is attached to an external pacemaker at 620. In one embodiment, the pacing protocol module includes a storage medium and an interface for connecting to an external pacemaker such as a PSA. With the pacing protocol module connected, the external pacemaker is capable of automatically executing the pacing protocol. An example of a pacing system including the pacing protocol module and the external pacemaker is discussed below, with reference to FIGS. 7-14.

Pacing electrodes are provided for use during the cardiac catheterization or surgery at 630. The pacing electrodes includes one or more pacing electrodes incorporated onto one or more PTVI devices and/or one or more cardiac surgical instruments as discussed in this document. In various embodiments, the one or more PTVI devices includes one or more of the IVUS catheter discussed in this document and other PTVI devices such as those discussed in U.S. patent application Ser. No. 11/113,828, entitled “METHOD AND APPARATUS FOR PACING DURING REVASCULARIZATION”, filed on Apr. 25, 2005, and U.S. Provisional Patent Application Ser. No. 61/074,066, entitled “EXTERNAL PACEMAKER WITH AUTOMATIC CARDIOPROTECTIVE PACING PROTOCOL”, filed on Jun. 19, 2008, both assigned to Cardiac Pacemakers, Inc., which are hereby incorporated by reference in their entirety. In one embodiment, the pacing electrodes also include additional one or more pacing electrodes not incorporated onto a PTVI device or cardiac surgical instrument, such as implantable electrodes in the patient and surface electrodes for attachment onto the patient's skin.

The delivery of the pacing pulses is controlled by automatically executing the instructions at 640, using the pacing system including the pacing protocol module and the external pacemaker. The pacing pulses are delivered via the pacing electrodes at 650.

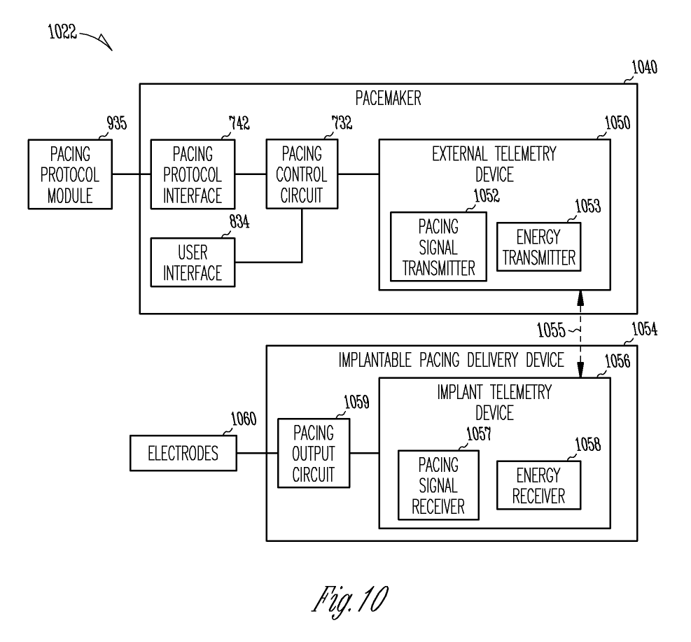

FIG. 7 is a block diagram illustrating of an embodiment of an external pacemaker 722, which is another embodiment of external pacemaker 322. External pacemaker 722 includes a pacemaker 740 and a pacing protocol module 735. Pacemaker 740 includes a pacing protocol interface 742 and a pacing control circuit 732. Pacing protocol interface 742 receives machine-readable instructions for automatically executing a pacing protocol. Pacing control circuit 732 controls delivery of pacing pulses by automatically executing the pacing protocol according to the received machine-readable instructions. In one embodiment, as further discussed with reference to FIGS. 11 and 12, pacing control circuit 732 is housed in a pacemaker chassis. Pacing protocol module 735 is external to the pacemaker chassis and is configured to be attached to pacemaker 740 and electrically connected to pacing protocol interface 742. Pacing protocol module 735 includes a storage device 743 that contains the machine-readable instructions for automatically executing the pacing protocol. In various embodiments, the pacing protocol specifies one or more of the cardioprotective pacing sequences illustrated in, and discussed above with reference to, FIGS. 4 and 5. In one embodiment, as further discussed with reference to FIGS. 11 and 12, storage device 743 is housed in a protocol chassis.

In various embodiments, the pacing protocol includes a therapy-specific pacing protocol that defines a pacing algorithm for treating a specific cardiac condition. In one embodiment, the pacing protocol provides for control of delivery of a pacing therapy through one or more PTVI devices such as those discussed in this document. The pacing protocol is a cardioprotective pacing protocol such as one of the cardioprotective pacing protocols discussed above with reference to FIG. 4 and FIG. 5. The cardioprotective pacing protocol provides for control of an acute pacing cardioprotection therapy during a cardiac catheterization or surgery procedure. In another embodiment, the pacing protocol provides for evaluation or optimization of pacing parameters during a device implantation procedure. An example of such a pacing protocol is a cardiac resynchronization therapy (CRT) protocol that provides for optimization of pacing parameters for CRT during implantation of a cardiac rhythm management device capable of delivering CRT. Another example of such a pacing protocol is a cardiac remodeling control therapy (RCT) protocol that provides for optimization of pacing parameters for RCT during implantation of a cardiac rhythm management device capable of delivering RCT. In one embodiment, the pacing protocol is a patient-specific pacing protocol created for an individual patient using one or more parameters indicative of the patient's cardiac condition.

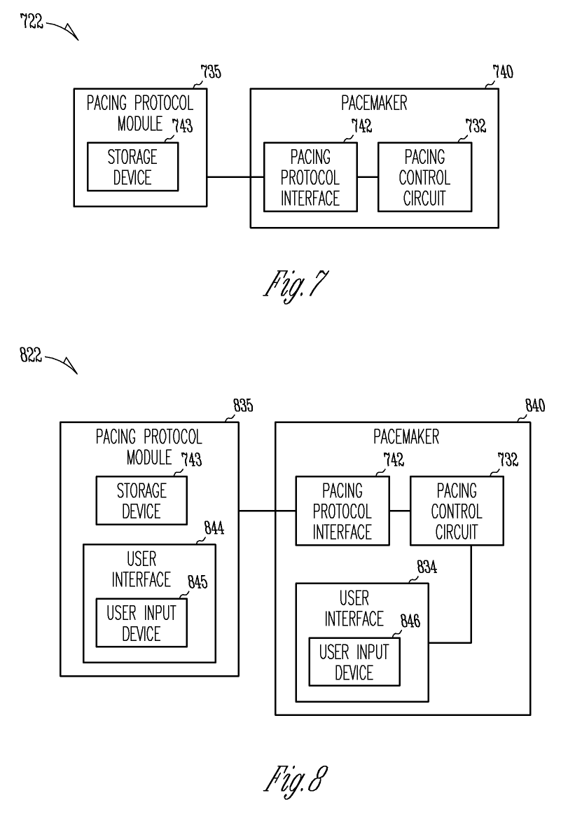

FIG. 8 is a block diagram illustrating of an embodiment of an external pacemaker 822, which is another embodiment of external pacemaker 722. External pacemaker 822 includes a pacemaker 840 and a pacing protocol module 835. Pacemaker 840 is another embodiment of pacemaker 740 and includes pacing protocol interface 742, pacing control circuit 732, and a pacemaker user interface 834. User interface 834 includes a user input device 846 that allows a user such as a physician or other caregiver to adjust user-adjustable pacing parameters of the pacing protocol. Pacing protocol module 835 is another embodiment of pacing protocol module 735. In the illustrated embodiment, pacing protocol module 835 includes storage device 743 and protocol user interface 844. User interface 844 includes a user input device 845 that allows the user to adjust user-adjustable pacing parameters of the pacing protocol. In another embodiment, pacing protocol module 835 does not include a user interface, and all the user-adjustable pacing parameters are adjusted using user interface 834 of pacemaker 840. In various embodiments, external pacemaker 822 includes one or both of user interfaces 845 and 846.

In one embodiment, pacemaker 840 includes a pacemaker chassis that houses at least pacing control circuit 732. In one embodiment, portions of pacing protocol interface 742 and user interface 834, including user input device 846, are mounted on the pacemaker chassis. In one embodiment, pacing protocol module 835 includes a protocol chassis that houses at least storage device 743. In one embodiment, portions of user interface 844, including user input device 845, are mounted on the protocol chassis.

FIG. 9 is a block diagram illustrating of an embodiment of a pacing system including an external pacemaker 922 connected to electrodes. External pacemaker 922 is another embodiment of external pacemaker 722 and includes a pacemaker 940 and a pacing protocol module 935. Pacemaker 940 is another embodiment of pacemaker 740 and includes pacing protocol interface 742, a pacing control circuit 936, user interface 834, a pacing output circuit 930, and a defibrillation output circuit 948. Pacing control circuit 936 controls delivery of cardioversion/defibrillation shocks in addition to performing the functions of pacing control circuit 732. Pacing output circuit 948 delivers pacing pulses through at least one of electrode(s) 949 of PTVI device(s) or surgical instrument(s) 910. Examples of electrode(s) 949 include the electrodes incorporated onto the IVUS catheter, heart stabilizer, and/or sternal retractor as discussed in this document. Defibrillation output circuit 948 delivers cardioversion/defibrillation shocks through at least one of electrode(s) 949. In one embodiment, a surface electrode 919 attached to the skin of the patient is also used for delivering the pacing pulses and/or cardioversion/defibrillation shocks. Pacing protocol module 935 includes pacing protocol module 735 or 835.

In one embodiment, pacemaker 940 is a PSA including a pacemaker chassis that houses at least pacing control circuit 936, pacing output circuit 930, and defibrillation output circuit 948. In one embodiment, portions of pacing protocol interface 742 and user interface 834, including user input device 846, are mounted on the pacemaker chassis.

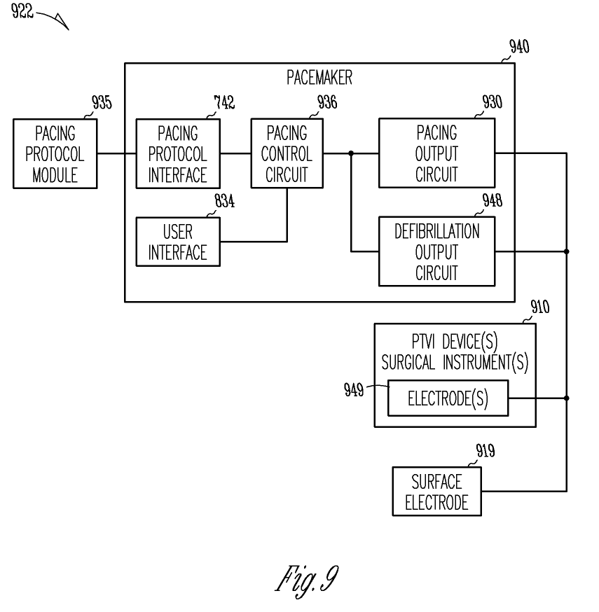

FIG. 10 is a block diagram illustrating of an embodiment of a pacing system including an external pacemaker 1022 and an implantable pacing delivery device 1054 connected to electrodes 1060. External pacemaker 1022 is another embodiment of external pacemaker 722 and includes a pacemaker 1040 and pacing protocol module 935. Pacemaker 1040 is another embodiment of pacemaker 740 and includes pacing protocol interface 742, pacing control circuit 732, user interface 834, and an external telemetry device 1050. Implantable pacing delivery device 1054 includes a pacing output circuit 1059 and an implant telemetry device 1056. Pacing output circuit 1059 delivers the pacing pulses through electrodes 1060 in response to pacing signals generated by pacing control circuit 732 and transmitted via a telemetry link 1055 supported by external telemetry device 1050 and implant telemetry device 1056. Electrodes 1060 includes pacing electrodes incorporated onto implantable pacing delivery device 1054 or electrically connected to implantable pacing delivery device 1054 through one or more implantable pacing leads.

In the illustrated embodiment, telemetry link 1055 is an inductive couple capable of transcutaneous signal and energy transmission. External telemetry device 1050 includes a pacing signal transmitter 1052 and an energy transmitter 1053. Pacing signal transmitter 1052 transmits the pacing signals for controlling the delivery of the pacing pulses. Energy transmitter 1053 transmits the energy required for implantable pacing delivery device 1054 to deliver the pacing pulses. Implant telemetry device 1056 includes a pacing signal receiver 1057 and an energy receiver 1058. Pacing signal receiver 1057 receives the pacing signals transmitted from pacing signal transmitter 1052. Energy receiver 1058 receives the energy transmitted from energy transmitter 1053.

In one embodiment, pacemaker 1040 includes a pacemaker chassis that houses at least pacing control circuit 732 and external telemetry device 1050. In one embodiment, portions of pacing protocol interface 742 and user interface 834, including user input device 846, are mounted on the pacemaker chassis.

In one embodiment, implantable pacing delivery device 1054 is implanted during the cardiac catheterization or surgery. In a specific embodiment, implantable pacing delivery device 1054 is integrated with a stent that is to be implanted during a PTCA procedure. External pacemaker 1022 and implantable pacing delivery device 1054 allow for delivery of an acute cardioprotective pacing therapy (also referred to an pacing postconditioning therapy) during the PTCA procedure after the stent is implanted, as well as a chronic cardioprotective pacing therapy (also referred to as intermittent pacing therapy) following the PTCA procedure.

FIG. 11 is an illustration of an embodiment of exterior configuration of an external pacemaker 1122 including a pacemaker 1140 and a pacing protocol module 1135. Examples of pacemaker 1140 include pacemakers 740, 840, 940, and 1040 as discussed above. An example of pacing protocol module 1135 includes pacing protocol module 835.

In the illustrated embodiment, pacemaker 1140 includes a pacemaker chassis 1165 housing its circuitry and portions of a pacemaker user interface 1132 mounted on pacemaker chassis 1165. Pacing protocol module 1135 includes a protocol chassis 1162 housing its circuitry and portions of a protocol user interface 1144 mounted on protocol chassis 1162. Pacing protocol module 1135 is attached to pacemaker 1140. In one embodiment, pacing protocol module 1135 is detachably attached to pacemaker 1140. This allows pacemaker 1140 to execute various pacing protocols by providing pacing protocol modules 1135 each storing one of the pacing protocols.

FIG. 12 is an illustration of an embodiment of exterior configuration of an external pacemaker 1222 including a pacemaker 1240 and a pacing protocol module 1235. Examples of pacemaker 1240 include pacemakers 740, 840, 940, and 1040 as discussed above. An example of pacing protocol module 1235 includes pacing protocol module 735.

In the illustrated embodiment, pacemaker 1240 includes a pacemaker chassis 1265 housing its circuitry and portions of a pacemaker user interface 1232 and a pacemaker connector 1264 mounted on pacemaker chassis 1265. Pacing protocol module 1235 includes a protocol chassis 1262 housing its circuitry and a protocol connector 1263 mounted on protocol chassis 1262. Pacing protocol module 1235 is configured as a plug-in module to be detachably attached to pacemaker 1240 by mating protocol connector 1263 with pacemaker connector 1264.

FIGS. 11 and 12 show examples of the external pacemaker for illustrative purposes. In various embodiments, the pacemaker and the pacing protocol module as discussed in this document have various exterior configurations. In embodiments illustrated in FIGS. 11 and 12, the pacing protocol module is externally attached to the pacemaker. In other embodiments, the pacing protocol module is also housed in the pacemaker chassis. In various embodiments, the pacing protocol module is configured in the forms of a plug-in module, a printed circuit board, a memory card, or an integrated circuit chip, that is detachably or non-detachably connected to the pacemaker to allow the pacemaker to execute one or more pacing protocols automatically.

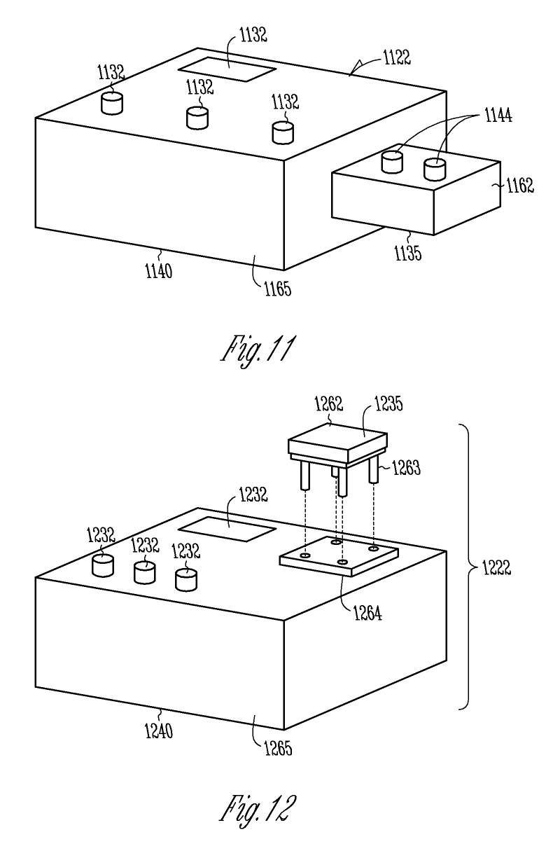

FIG. 13 is a block diagram of an embodiment of exterior configuration of an external device 1325, which includes a pull back motor 1324 integrated pacemaker 1140. External device 1325 represents an embodiment of the exterior configuration of external device 125. Pull back motor 1324 represents an embodiment of pull back motor 124 and is housed within chassis 1165.

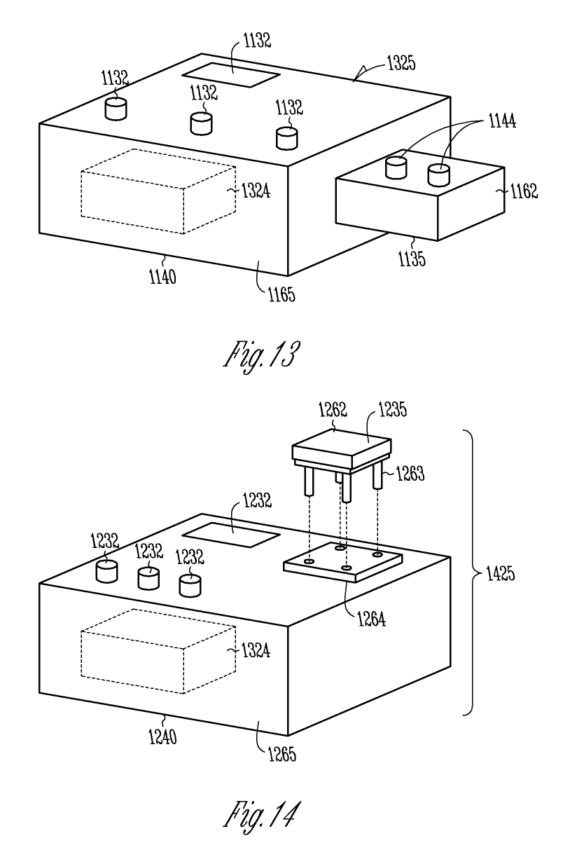

FIG. 14 is a block diagram of an embodiment of exterior configuration of an external device 1425, which includes pull back motor 1324 integrated pacemaker 1240. External device 1425 represents another embodiment of the exterior configuration of external device 125. Pull back motor 1324 represents an embodiment of pull back motor 124 and is housed within chassis 1265.

In one embodiment, instead of using a stand-alone external pacemaker, a pacemaker is integrated into a device or instrument used in the cardiac catheterization or surgery. An example of a pacemaker suitable for being integrated into an IVUS catheter or a cardiac surgical instrument discussed in this document is discussed in U.S. Provisional Patent Application Ser. No. 61/074,048, entitled “PACEMAKER INTEGRATED WITH VASCULAR INTERVENTION CATHETER”, filed on Jun. 19, 2008, both assigned to Cardiac Pacemakers, Inc., which is hereby incorporated by reference in its entirety. The pacemaker is capable of delivering pacing pulses by automatically executing a pacing protocol such as one of the cardioprotective pacing protocols discussed above with reference to FIG. 4 and FIG. 5.

### IVUS Catheter With Pacing Electrode(s)

IVUS imaging provides for visualization inside a blood vessel, for example, during revascularization to assess plaque built-up in the blood vessel as well as result of angioplasty. An IVUS imaging catheter with one or more pacing electrodes allows for delivery of cardioprotective pacing during the revascularization procedure. In the discussion below, an “IVUS catheter” refers to an IVUS catheter including one or more pacing electrodes.

In an example of application, cardioprotective pacing is delivered during a PCTA procedure performed on a patient having suffered acute MI. A pacing post-conditioning therapy is delivered during reperfusion that follows the reopening of the coronary artery in which angioplasty has been performed. In one embodiment, the patient also receives a long-term intermittent pacing therapy after the PCTA procedure. The intermittent pacing therapy includes periodic delivery of a cardioprotective pacing sequence that is identical or substantially similar to the cardioprotective pacing sequence specified in the cardioprotective pacing protocol illustrated in, and discussed above with reference to, FIGS. 4 and 5. In one embodiment, after a stent is placed in the coronary artery, an IVUS catheter is inserted to determine stent apposition to the vessel wall. Pacing pulses are delivered through the IVUS catheter according to the cardioprotective pacing protocol. Execution of the cardioprotective pacing protocol is initiated in response to an indication of reperfusion. In one embodiment, anti-arrhythmic pacing is also delivered to prevent or treat arrhythmia or electrical cardiac disturbances during the PCTA procedure. In one embodiment, one or more cardiac signals are sensed using electrodes on the IVUS catheter and/or other electrodes for detection of the arrhythmia or electrical cardiac disturbances. In one embodiment, the IVUS catheter also includes one or more electrodes suitable for delivering cardioversion/defibrillation pulses. This allows timely application of a cardioversion/defibrillation therapy when necessary.

In various embodiments, pacing pulses are delivered according to the cardioprotective pacing protocol using one or more PTVI devices used during a revascularization procedure, including the IVUS catheter, based on desirable timing of therapy delivery. The pacing reduces the extent of myocardial injury associated with MI as well as the revascularization procedure. Integration of one or more pacing electrodes into such PTVI devices allows cardioprotective pacing to be delivered without substantially prolonging the revascularization procedure.

FIG. 15 is an illustration of an embodiment of an IVUS catheter 1510 that allows for delivery of pacing pulses. IVUS catheter 1510 is an embodiment of IVUS catheter 110 and includes a proximal end portion 1512, a distal end portion 1511 configured for intravascular placement, and an elongate shaft 1513 coupled between proximal end portion 1512 and distal end portion 1511. In various embodiments, one or more pacing electrodes are incorporated onto distal end portion 1511 and/or shaft 1513.

Proximal end portion 1512 includes a catheter connector 1577 and an injection port 1572. Catheter connector 1571 includes one or more connectors configured to provide mechanical and electrical connections between IVUS catheter 1510 and external pacemaker 122, pullback motor 124, and ultrasound machine 109. In the illustrated embodiment, catheter connector 1577 is configured to provide the mechanical and electrical connections with a single physical connection between catheter connector 1577 and external device 125, which includes integrated external pacemaker 122 and pullback motor 124 with a single connector integrating pacemaker connector 123 and motor connector 126. Catheter connector 1577 includes a transducer connector 1514 that mates motor connector 126, pacing connectors (contacts) 1516A-B that contact pacemaker connector 123 for electrical connection to external pacemaker 122, and an ultrasound connector (one or more contacts) 1570 for electrical connection to ultrasound machine 109. Injection port 1572 allows for injection of a liquid from liquid source 117. Examples of the liquid include saline, drugs, and liquid agents that enhance the ultrasound image.

Distal end portion 1511 includes an ultrasonic transducer 1567, pacing electrodes 1574A-B, and an exit port 1571. Ultrasonic transducer 1567 transmits an ultrasound signal and receives an image signal related to the transmitted ultrasound signal. Pacing electrodes 1574A-B allow for delivery of the pacing pulses. For illustrative purposes, two pacing electrodes are shown in FIG. 15. In various embodiments, distal end portion 1511 includes one, two, or more pacing electrodes. In one embodiment, distal end portion 1511 includes at least one electrode for a unipolar pacing configuration with another electrode. In another embodiment, distal end portion 1511 includes at least two electrodes for a bipolar pacing configuration. In one embodiment, one or more additional pacing electrodes are incorporated onto shaft 1513. In one embodiment, one or more of the pacing electrodes are configured to allow delivery of cardioversion/defibrillation pulses. Exit port 1571 allow exit of the liquid from IVUS catheter 1510.

Mechanical and electrical links extend in shaft 1513. A rotating drive shaft 1568 is connected between ultrasonic transducer 1567 and transducer connector 1514 to allow ultrasonic transducer 1567 to be driven by pullback motor 124. An ultrasound lead 1569 is connected between ultrasonic transducer 1567 and ultrasound connector 1570 and includes multiple conductors to transmit signals to and from ultrasonic transducer 1567. Pacing leads 1575A-B are connected between pacing electrodes 1574A-B and pacing connectors 1516A-B to conduct the pacing pulses. A lumen 1573 connects injection port 1572 and exit port 1571 to allow the liquid to flow through IVUS catheter 1510. In one embodiment, lumen 1573 also accommodates a portion of a guide wire used to guide the insertion of IVUS catheter 1510. Rotating drive shaft 1568, ultrasound lead 1569, pacing leads 1575A-B, and lumen 1572 are shown in FIG. 15 to illustrate connections between components without necessarily reflecting their physical appearance and relative positions.

In one embodiment, IVUS catheter 1510 is made as a disposable device. In one embodiment, the design of IVUS catheter 1510 is based on an existing IVUS catheter, and modification to the design is made to incorporate the one or more pacing electrodes. One example of the existing IVUS catheter is the Atlantis® SR Pro Coronary Imaging Catheter provided by Boston Scientific Corporation.

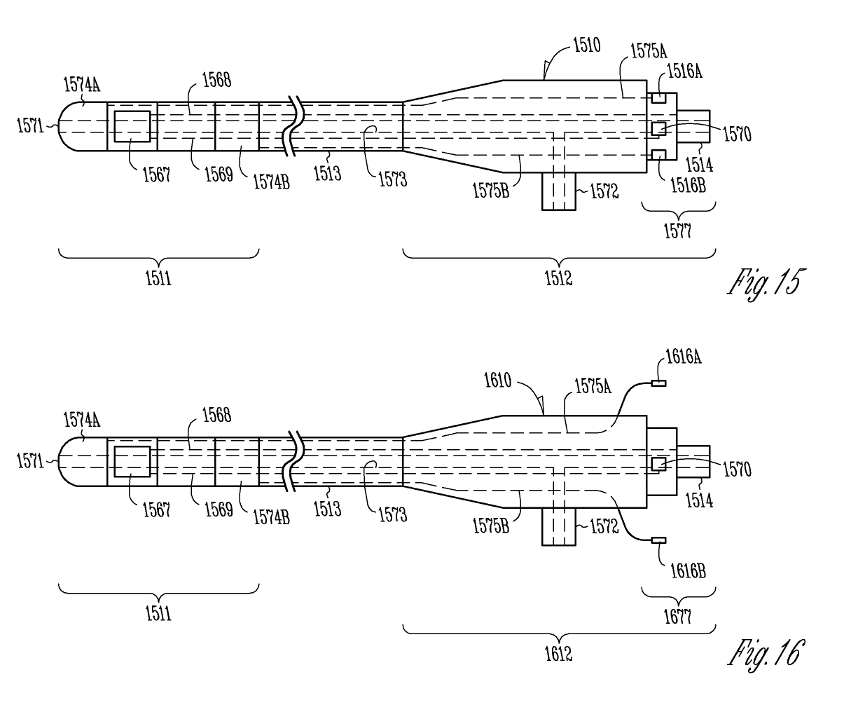

FIG. 16 is an illustration of an embodiment of an IVUS catheter 1610 that allows for delivery of pacing pulses. IVUS catheter 1610 is another embodiment of IVUS catheter 110 and includes a proximal end portion 1612, distal end portion 1511 configured for intravascular placement, and elongate shaft 1513. IVUS catheter 1610 is substantially identical to IVUS catheter 1510 except for that proximal end 1612 includes a catheter connector 1677, which differs from catheter 1577 of proximal end 1512.

In the illustrated embodiment, catheter connector 1677 is configured to make the mechanical and electrical connections by multiple physical connections between catheter connector 1677 and external device 125. Catheter connector 1677 includes transducer connector 1514 that mates motor connector 126, pacing connectors 1616A-B that branches out for connections to external pacemaker 122, and an ultrasound connector (one or more contacts) 1570 for electrical connection to ultrasound machine 109. The configuration of catheter connector 1677 is suitable, for example, when external pacemaker 122 and pullback motor 124 are physically separate devices each having its own chassis.

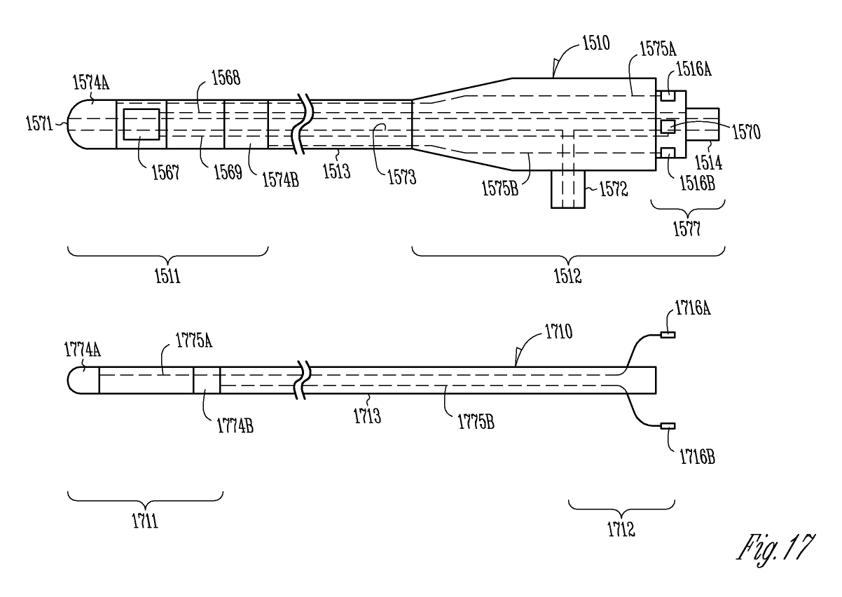

FIG. 17 is an illustration of an embodiment of a catheterization device assembly allowing for delivering pacing pulses during cardiac catheterization, including IVUS catheter 1510 and another PTVI device 1710. PTVI device 1710 represent a device such as a guide wire, a guide catheter, or an angioplasty catheter that is to be placed in the patient's body concurrently with ITVS catheter 1510. Examples of PTVI device 1710 includes those discussed in U.S. patent application Ser. No. 11/113,828 and U.S. Provisional Patent Application Ser. No. 61/074,066.

PTVI device 1710 includes a proximal end portion 1712, a distal end portion 1711 configured for intravascular placement, and an elongate shaft 1713 coupled between proximal end portion 1712 and distal end portion 1711. Distal end portion 1711 includes pacing electrodes 1774A-B that allow for delivery of pacing pulses. Two pacing electrodes are shown in FIG. 17 for illustrative purpose only. In various embodiments, distal end portion 1711 includes one, two, or more pacing electrodes. In one embodiment, one or more additional pacing electrodes are incorporated onto shaft 1713. Proximal end portion 1712 includes pacing connectors 1716A-B configured to be electrical connected to external pacemaker 122. Pacing leads 1775A-B extend within or over shaft 1713 and are connected between pacing electrodes 1774A-B and pacing connectors 1716A-B to conduct the pacing pulses. In one embodiment, one or more of the pacing electrodes are configured to allow delivery of cardioversion/defibrillation pulses. In various embodiments, pacing pulses are delivered using a pair of electrodes selected from pacing electrodes 1574A-B and 1774A-B. In one embodiment, PTVI device 1710 represents a guide wire over which IVUS catheter 1510 is inserted into the vascular system of the patient.

IVUS catheter 1510 and PTVI device 1710 are shown in FIG. 17 for illustrative purposes. In one embodiment, the catheterization device assembly includes IVUS catheter 1610 instead of IVUS catheter 1510. In various embodiments, the catheterization device assembly includes any number and types of PTVI devices onto which one or more pacing electrodes are incorporated.

In various embodiments, pacing pulses are delivered using one or more pairs of pacing electrodes selected from the one or more pacing electrodes of an IVUS catheter, one or more pacing electrodes of other one or more PTVI devices, a body-surface electrode, and one or more electrodes of an implantable medical device. The selection of each electrode pair depends on, among other things, the availability and the location of each electrode in the patient's body when pacing is to be delivered.

### Heart Stabilizer With Pacing Electrode(s)

A heart stabilizer holds the heart in a desirable position using vacuum suction during an open-chest heart surgery such that the surgeon can operate on or about the heart while it is beating. In one example, the heart stabilizer is clamped on a sternal retractor, which retracts the patient's rib cage to keep the chest open and the heart exposed. A heart stabilizer with one or more pacing electrodes allows for delivery of cardioprotective pacing during the cardiac surgery. When desirable, the sternal retractor functions as a return electrode. In the discussion below, a “heart stabilizer” refers to a heart stabilizer with one or more pacing electrodes.

In an example of application, cardioprotective pacing is delivered during a CABG surgery performed on a patient with a substantially blocked coronary artery. The heart stabilizer is placed on the myocardium when it is accessible. One or more pacing electrodes on the heart stabilizer are placed adequately, in good contact with myocardial tissue in the intended pacing sites. Pacing cardioprotection therapies are delivered through the heart stabilizer (and the sternal retractor if desirable) before starting the CABG procedure for preconditioning the myocardium and after the grafting is completed for postconditioning the myocardium. In one embodiment, execution of the cardioprotective pacing protocol is initiated in response to an indication of reperfusion after the CABG procedure is completed. In one embodiment, anti-arrhythmic pacing is also delivered to prevent or treat arrhythmia or electrical cardiac disturbances during the surgery. In one embodiment, one or more cardiac signals are sensed using electrodes on the heart stabilizer catheter and/or other electrodes for detection of the arrhythmia or electrical cardiac disturbances. In one embodiment, the heart stabilizer also includes one or more electrodes suitable for delivering cardioversion/defibrillation pulses. This allows timely application of a cardioversion/defibrillation therapy when necessary.

In various embodiments, pacing pulses are delivered by executing the cardioprotective pacing protocol using one or more surgical instruments attached to the patient during the cardiac surgery procedure based on desirable timing of therapy delivery. The pacing reduces the extent of myocardial injury associated with the surgical procedure. Integration of one or more pacing electrodes into surgical instruments allows cardioprotective pacing to be delivered without substantially prolonging the surgical procedure.

FIG. 18 is an illustration of an embodiment of a heart stabilizer 1827 that allow for delivery of pacing pulses. Heart stabilizer 1827 is an embodiment of heart stabilizer 127 and includes a suction cup 1885, an elongate vacuum tube 1878 coupled to suction cup 1885, a positioning handle 1884, a positioning arm 1880 coupled between suction cup 1885 and positioning handle 1884, and a base 1889 coupled to positioning arm 1880. In various embodiments, one or more pacing electrodes are incorporated onto one or more locations of suction cup 1885.

Suction cup 1885 is configured to hold the patient's heart using vacuum suction provided by vacuum source 215 through vacuum tube 1878. It includes a surface portion 1886 that is to be in direct contact with the heart during use. In one embodiment, suction cup 1885 is made of a flexible material such as silicone. In the illustrated embodiment, two groups of pacing electrodes 1876A-B are affixed onto surface portion 1886. The electrodes are grouped, for example, to ensure that each group provides at least one reliable electrical connection to the heart. In various embodiments, one or more pacing electrodes, or one or more groups of pacing electrodes, are affixed onto surface portion 1886, depending on the anticipated need for effective and reliable pacing delivery during the cardiac surgery. In one embodiment, at least two electrodes, or at least two groups of electrodes, are affixed onto surface portion 1886 for a bipolar pacing configuration. In another embodiment, at least one electrode, or at least one group of electrodes, is affixed onto surface portion 1886 for a unipolar pacing configuration. The return electrode for the unipolar pacing configuration includes one of a body-surface electrode and an electrode being a portion of another cardiac surgical instrument such as the sternal retractor. In one embodiment, one or more of the pacing electrodes are configured to allow delivery of cardioversion/defibrillation pulses.

Positioning arm 1880 includes a distal end 1883 connected to suction cup 1885, a proximal end 1882 connected to positioning handle 1884, and an elongate positioning shaft 1881 connected between distal end 1883 and the proximal end 1882. Positioning handle 1884 allows a user to manipulate the position of suction cup 1885 by adjusting the shape of positioning shaft 1881.

Pacing leads 1875A-B electrically connect pacing electrodes 1876A-B to pacing connectors 1816A-B. Pacing connectors 1816A-B are configured to be connected to external pacemaker 122. In the illustrated embodiment, pacing lead 1875A-B extend within a portion of positioning shaft 1881. In another embodiment, pacing lead 1875A-B extend over a portion of positioning shaft 1881. In another embodiment, pacing lead 1875A-B extend within or over vacuum tube 1878.

Base 1889 is connected to positioning shaft 1881 near proximal end 1882 for stabilizing the position of heart stabilizer 1827 relative to the body of the patient. In one embodiment, base 1889 is configured to be clamped on a cardiac surgical instrument such as the sternal retractor.

In one embodiment, the design of heart stabilizer 1827 is based on an existing heart stabilizer, and modification to the design is made to incorporate the one or more pacing electrodes. One example of the existing heart stabilizer is the EPOSET® 3 Access Device provided by Boston Scientific Corporation.

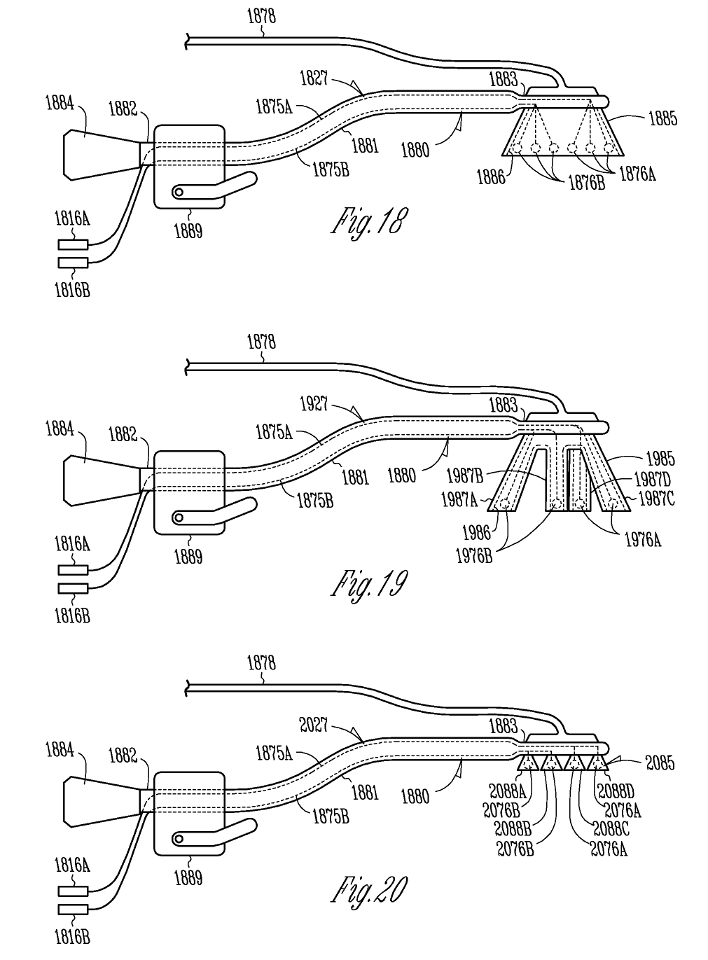

FIG. 19 is an illustration of an embodiment of a heart stabilizer 1927 that allows for delivery of pacing pulses. Heart stabilizer 1927 is another embodiment of heart stabilizer 127 and is substantially identical to heart stabilizer 1827 except for a suction cup 1985 that has a configuration different from that of suction cup 1885. Suction cup 1985 is a multi-appendage suction cup. In various embodiments, one or more pacing electrodes are incorporated onto one or more appendages of suction cup 1985.

As shown in FIG. 19 for illustrative purposes, suction cup 1985 includes appendages 1987A-D for smaller area of direct contact between the heart and suction cup 1987 to enhance visualization of the heart under suction cup 1987. Each of appendages 1987A-D includes a surface portion 1986 that is to be in direct contact with the heart during use. In the illustrated embodiment, two groups of pacing electrodes 1976A-B are affixed onto surface portions 1986 of appendages 1987A-D. In various embodiments, suction cup 1985 includes two or more appendages. One or more pacing electrodes, or one or more groups of pacing electrodes, are affixed onto one or more surface portions of one or more of the appendages, depending on the anticipated need for effective and reliable pacing delivery during cardiac surgery.

FIG. 20 is an illustration of an embodiment of a heart stabilizer 2027 that allows for delivery of pacing pulses. Heart stabilizer 2027 is another embodiment of heart stabilizer 127 and is substantially identical to heart stabilizer 1827 except for a suction cup 2085 that has a configuration different from that of suction cup 1885. Suction cup 2085 includes an array of small suction cups. In various embodiments, one or more pacing electrodes are incorporated onto one or more of the small suction cups.

As shown in FIG. 20 for illustrative purposes, suction cup 2085 includes small suction cups 2088A-D. Two groups of pacing electrodes 2076A-B are affixed onto inner surface portions of appendages 1987A-D. In various embodiments, one or more pacing electrodes, or one or more groups of pacing electrodes, are affixed onto one or more inner surface portions of one or more of small suction cups, depending on the anticipated need for effective and reliable pacing delivery during cardiac surgery.

Heart stabilizers 1827, 1927, and 2027 are shown in FIGS. 18-20 for illustrative purposes only. In various embodiments, one or more pacing electrodes are incorporated into one or more suction cups of a heart stabilizer in a way ensuring reliable electrical contact with the heart for delivering pacing pulses.

It is to be understood that the above detailed description, including the various examples of IVUS catheters, heart stabilizers, and external pacemakers, is intended to be illustrative, and not restrictive. In general, cardioprotective pacing is applied to prevent or reduce cardiac injury associated with ischemia and reperfusion by using one or more pacing electrodes incorporated onto any cardiac catheterization or surgical device and a pacemaker that is capable of delivering pacing pulses by executing a cardioprotective pacing protocol. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A pacing system for use during a percutaneous transluminal vascular intervention (PTVI) procedure, the system comprising:
an intravascular ultrasound (IVUS) catheter being a PTVI device including a proximal end portion, a distal end portion, and an elongate shaft connected between the proximal end portion and the distal end portion, the distal end portion configured for intravascular placement and including:
an ultrasonic transducer configured to transmit an ultrasound signal and receive an image signal related to the transmitted ultrasound signal; and
a first pacing electrode configured to deliver pacing pulses.

2. The system of claim 1, comprising an external device including:
a pullback motor;
an external pacemaker; and
a chassis housing the pullback motor and the external pacemaker,
and wherein the proximal end portion of the IVUS catheter comprises a catheter connector including one or more connectors configured to provide connections each between the IVUS catheter and one of the pullback motor and the external pacemaker.

3. The system of claim 1, comprising:
a pacing output circuit configured to be connected to the proximal end portion of the IVUS catheter and deliver the pacing pulses to the first pacing electrode;
a pacing control circuit configured to control the delivery of the pacing pulses by automatically executing a cardioprotective pacing protocol specifying pacing parameters selected to augment cardiac mechanical stress to a level effecting cardioprotection against myocardial injury using the pacing pulses; and
a pacing protocol module coupled to the pacing control circuit, the pacing protocol module containing machine-readable instructions for automatically executing the cardioprotective pacing protocol.

4. The system of claim 3, comprising an external pacemaker including:
a pacemaker chassis housing the pacing output circuit and the pacing control circuit; and
a pacing protocol interface incorporated onto the chassis and configured to receive the machine-readable instructions from the pacing protocol module,
and wherein the pacing protocol module is configured to be externally attached to the external pacemaker and electrically connected to the pacing protocol interface.

5. The system of claim 3, wherein the distal end portion of the IVUS catheter comprises a second pacing electrode configured to allow the pacing pulses to be delivered using the first pacing electrode and the second pacing electrode.

6. The system of claim 3, comprising a body-surface electrode configured to be electrically connected to the pacing output circuit to allow the pacing pulses to be delivered using the first pacing electrode and the body-surface electrode.

7. The system of claim 3, comprising a further PTVI device other than the IVUS catheter, the further PTVI device configured to be electrically connected to the pacing output circuit and including a second pacing electrode configured to allow the pacing pulses to be delivered using the first pacing electrode and the second pacing electrode.

8. A method for cardiac pacing during a percutaneous transluminal vascular intervention (PTVI) procedure, the method comprising:
providing an intravascular ultrasound (IVUS) catheter being a PTVI device including a distal end portion configured for intravascular placement, the distal end portion including an ultrasonic transducer and a first pacing electrode; and
delivering pacing pulses through the first pacing electrode during the PTVI procedure.

9. The method of claim 8, comprising controlling the delivery of the pacing pulses by automatically executing a pacing protocol using an external pacemaker connected to the IVUS catheter.

10. The method of claim 9, wherein automatically executing the pacing protocol comprises automatically executing a cardioprotective pacing protocol adapted to augment cardiac mechanical stress to a level effecting cardioprotection against myocardial injury using the pacing pulses, the cardioprotective pacing protocol specifying a cardiac protection pacing sequence including alternating pacing and non-pacing periods, the pacing periods each having a pacing duration during which a plurality of the pacing pulses is delivered, the non-pacing periods each having a non-pacing duration during which none of the pacing pulses is delivered.

11. The method of claim 10, comprising providing the cardioprotective pacing protocol specifying a number of cycles of the alternating pacing and non-pacing periods between 1 and 4 cycles, the pacing duration between 10 seconds and 20 minutes, and the non-pacing duration between 10 seconds and 20 minutes.

12. The method of claim 11, comprising initiating the delivery of the pacing pulses by automatically executing the cardioprotective pacing protocol in response to an indication of reperfusion during the PTVI procedure.

13. A pacing system for use during a cardiac surgery performed on or about a heart, the system comprising:
a heart stabilizer including:
a suction cup configured to hold the heart using vacuum suction and including a first pacing electrode configured to deliver pacing pulses to the heart; and
a positioning arm including a distal end connected to the suction cup, a proximal end including a positioning handle, and an elongate positioning shaft connected between the distal end and the proximal end, the positioning handle configured to allow a user to manipulate the position of the suction cup by adjusting the shape of the positioning shaft.

14. The system of claim 13, wherein the suction cup comprises a plurality of pacing electrodes to deliver the pacing pulses, the plurality of pacing electrodes including the first pacing electrode.

15. The system of claim 14, wherein the suction cup comprises a multi-appendage suction cup including a plurality of appendages, and the plurality of pacing electrodes comprises pacing electrodes incorporated onto one or more appendages of the plurality of appendages.

16. The system of claim 14, wherein the suction cup comprises an array of small suction cups, and the plurality of pacing electrodes comprises pacing electrodes incorporated onto one or more small suction cups of the array of small suction cups.

17. The system of claim 13, comprising a sternal retractor including a second pacing electrode configured to allow the pacing pulses to be delivered through the first pacing electrode and the second pacing electrode, and wherein the heart stabilizer comprises a base connected to the positioning shaft and configured to be clamped on the sternal retractor.

18. The system of claim 13, comprising:
a pacing output circuit configured to be connected to the heart stabilizer and deliver the pacing pulses to the first pacing electrode;
a pacing control circuit configured to control the delivery of the pacing pulses by automatically executing a cardioprotective pacing protocol specifying pacing parameters selected to augment cardiac mechanical stress to a level effecting cardioprotection against myocardial injury using the pacing pulses; and
a pacing protocol module coupled to the pacing control circuit, the pacing protocol module containing machine-readable instructions for automatically executing the cardioprotective pacing protocol.

19. The system of claim 18, comprising an external pacemaker including:
a pacemaker chassis housing the pacing output circuit and the pacing control circuit; and
a pacing protocol interface incorporated onto the chassis and configured to receive the machine-readable instructions from the pacing protocol module,
and wherein the pacing protocol module is configured to be externally attached to the external pacemaker and electrically connected to the pacing protocol interface.

20. A method for cardiac pacing during cardiac surgery, the method comprising:
providing a heart stabilizer including a suction cup configured to hold the heart using vacuum suction and including a first pacing electrode; and
delivering pacing pulses through the first pacing electrode.

21. The method of claim 20, comprising:
providing a sternal retractor including a second pacing electrode; and
delivering the pacing pulses through the first pacing electrode and the second pacing electrode.

22. The method of claim 20, comprising controlling the delivery of the pacing pulses by automatically executing a pacing protocol using an external pacemaker connected to the heart stabilizer.

23. The method of claim 22, wherein automatically executing the pacing protocol comprises automatically executing a cardioprotective pacing protocol adapted to augment cardiac mechanical stress to a level effecting cardioprotection against myocardial injury using the pacing pulses, the cardioprotective pacing protocol specifying a cardiac protection pacing sequence including alternating pacing and non-pacing periods, the pacing periods each having a pacing duration during which a plurality of the pacing pulses is delivered, the non-pacing periods each having a non-pacing duration during which none of the pacing pulses is delivered.

24. The method of claim 23, comprising providing the cardioprotective pacing protocol specifying a number of cycles of the alternating pacing and non-pacing periods between 1 and 4 cycles, the pacing duration between 10 seconds and 20 minutes, and the non-pacing duration between 10 seconds and 20 minutes.

25. The method of claim 24, comprising initiating the delivery of the pacing pulses by automatically executing the cardioprotective pacing protocol in response to an indication of reperfusion during the cardiac surgery.
